SEQUENCE LISTING (23 sequences)

SEQ ID NO: 1 - protein, 594 aa, Aeromonas caviae
MSQPSYGPLFEALAHYNDKLLAMAKAQTERTAQALLQTNLDDLGQVLEQGSQQPWQLIQAQMNWWQDQLKLMQHTLLKSAGQPSEPVITPERSDRRFKAEAWSEQPIYDYLKQSYLLTARHLLASVDALEGVPQKSRERLRFFTRQYVNAMAPSNFLATNPELLKLTLESDGQNLVRGLALLAEDLERSADQLNIRLTDESAFELGRDLALTPGRVVQRTELYELIQYSPTTETVGKTPVLIVPPFINKYYIMDMRPQNSLVAWLVAQGQTVFMISWRNPGVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPAANKYGFWHNGAEAESPESWLAGATHQGGSWWPEMMGFIQNRDEGSEPVPARVPEEGLAPAPGHYVKVRLNPVFACPTEEDAA

SEQ ID NO: 2 - protein, 3 aa, Artificial Sequence
  Peroxisome Targetting Sequence
SKL

SEQ ID NO: 3 - protein, 3 aa, Artificial Sequence
  Peroxisome Targetting Sequence
AKI

SEQ ID NO: 4 - DNA, 9 bp, Artificial Sequence
  Peroxisome Targetting Sequence
tctaaattg

SEQ ID NO: 5 - DNA, 9 bp, Artificial Sequence
  Peroxisome Targetting Sequence
gctaaaatt

SEQ ID NO: 6 - DNA, 750 bp, Candida maltosa
  ALK5 Promoter
aagcttcacatggatcaattgcgtttgtcacatgtggtcatccagctatggttgatgaggttagatattttacttgtaagaatattaacaacccagaaaagaaaagagttgatttctttgaacaagtgcaagtctgggcttagacgtttatttttgtttttgttgagtggtaatacatattcttcgtatctatgaagatttttcacacgcggatagtaattgtactagccgcttctttaagtaactgatttacccaacaagtacatggtaatacaaactctcactcactagacttcgcttctagttgcttcaaattagacggttataatgtatgccaaggttttgtgtaatttcacggtgattaaccttttcccctttttatactcctcattatccacgatgtaatctgatctatgaacgtgataagtaacattacttagtcattaagtatggccaattcagttatacatattagtaatgctccacatccattgtattcatatgtaatgccaaatatcacattcatttacacagaatcggttttgttaaatactccgctattgtacagcaacaataggattatgtacagaatgaaaaacaaaaggcggagaaattcgacggaaaaatttattatttacaaatcgtattcccgcattatctataaaacagattcaaaataatctagatctcttttttttgcttccttttatttctttttaaataagattaaactaaaaatatgattgatga

SEQ ID NO: 7 - DNA, 73 bp, Candida maltosa
  ARR
catgtgcttttttttttgttttcaatttgaaagtttttttatttccgcaatacaaaattattttttatccgct

SEQ ID NO: 8 - DNA, 32 bp, Artificial Sequence
  Primer
gatggagccatagcggagacgtattgtctagt

SEQ ID NO: 9 - DNA, 32 bp, Artificial Sequence
  Primer
actagacaatacgtctccgctatggctccatc

SEQ ID NO: 10 - DNA, 22 bp, Artificial Sequence
  Primer
actaacttggatgatttgggtc

SEQ ID NO: 11 - DNA, 22 bp, Artificial Sequence
  Primer
ctaatttcttggctactaaccc

SEQ ID NO: 12 - DNA, 22 bp, Artificial Sequence
  Primer
agttttgatcgttccaccattc

SEQ ID NO: 13 - DNA, 20 bp, Artificial Sequence
  Primer
catgaaccaattatcgccgc

SEQ ID NO: 14 - DNA, 22 bp, Artificial Sequence
  Primer
gttggtttctgccgttgatgat

SEQ ID NO: 15 - DNA, 754 bp, Artificial Sequence
  ARR-Alk2 promoter
aagcttgcatgcctgcaggtcgaaattcgagctcggtacccggggatcctctagagtccatgtgcttttttttttgttttcaatttgaaagtttttttatttccgcaatacaaaattattttttatccgctcatgtgcttttttttttgttttcaatttgaaagtttttttatttccgcaatacaaaattattttttatccgctgacccagatcctctagagtccatgtgcttttttttttgttttcaatttgaaagtttttttatttccgcaatacaaaattattttttatccgctcatgtgcttttttttttgttttcaatttgaaagtttttttatttccgcaatacaaaattattttttatccgctgacccagatcctctagagtccatgtgcttttttttttgttttcaatttgaaagtttttttatttccgcaatacaaaattattttttatccgctcatgtgcttttttttttgttttcaatttgaaagtttttttatttccgcaatacaaaattattttttatccgctgacccagatctcgactctagaggatccccgtttttttatttccgcaatacaaaattattttttatccgctttccgttcctttcttcttgtgataaatctcaacaattatatatatcattccataaccctgaataattttttttttaagtccttggtttctttttttagaaaaaaaggtgaatcagtaaaatttttgttatttatcattttaactcaca

SEQ ID NO: 16 - DNA, 83 bp, Artificial Sequence
  Synthetic DNA
gtccttggtttctttttttagaaaaaaaggtgaatcagtaaaatttttgttatttatcattttaactcacatatgaagatatc

SEQ ID NO: 17 - DNA, 45 bp, Artificial Sequence
  primer
atggtactgcagttacaatttagaagcagcatcttcttcggttgg

SEQ ID NO: 18 - DNA, 99 bp, Artificial Sequence
  Primer
acacatatgtctcaaccatcttatggtccattgttcgaagctttggctcattacaatgataaattgttggctatggctaaagctcaaaccgaaagaact

SEQ ID NO: 19 - DNA, 29 bp, Artificial Sequence
  Primer
gactttggaatccggtggtcaaaatttgg

SEQ ID NO: 20 - DNA, 29 bp, Artificial Sequence
  Primer
ccaaattttgaccaccggattccaaagtc

SEQ ID NO: 21 - DNA, 340 bp, Kluyveromyces lactis
  LACt
gaattttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatca

SEQ ID NO: 22 - DNA, 40 bp, Artificial Sequence
  primer
gcgcctgcaggaattttatacttagataagtatgtactta

SEQ ID NO: 23 - DNA, 40 bp, Artificial Sequence
  primer
ggcgtcgacaagcttgtttctgagcatgctgaaattttct

(19) United States
(12) Patent Application Publication
(10) Pub. No.: US 2007/0087421 A1
(43) Pub. Date: Apr. 19, 2007

(54) Transformant and process for producing polyester using the same

(76) Inventors: Yuji Okubo, Takasago-shi (JP); Tetsuya Nagaoka, Kobe-shi (JP); Satoru Yokomizo, Akashi-shi (JP); Keiji Matsumoto, Nishinomiya-shi (JP); Masamichi Takagi, Fuchu-shi (JP); Akinori Ohta, Saitama-shi (JP)

Correspondence Address: KENYON & KENYON LLP, 1500 K STREET N.W., SUITE 700, WASHINGTON, DC, 20005, US

(21) Appl. No.: 10/554,934

(22) PCT Filed: May 14, 2004
(86) PCT No.: PCT/JP04/06542

§ 371 Date: Sep. 14, 2006

(30) Foreign Application Priority Data
May 15, 2003 (JP) 2003-137492

(51) Int. Cl. C12P 7/62; C12N 9/18; C07H 21/04; C12N 15/74; C12N 1/21

(52) U.S. Cl. 435/135; 435/197; 435/252.3; 435/471; 536/23.2

(57) ABSTRACT

A gene expression cassette comprising an Aeromonas caviae-origin polyhydroxyalkanoic acid (PHA) synthase gene having a mutation introduced thereinto and a promoter and a terminator acting in a yeast; a transformant constructed by transferring the above gene expression cassette into a yeast; and a process for producing a polyester using the above transformant. Thus, it becomes possible to efficiently produce a copolymerized polyester having a high biodegradability and excellent properties by copolymerizing 3-hydroxyalkanoic acids in a yeast.

### TECHNICAL FIELD

The present invention relates to genes necessary for the enzymatic synthesis of copolyesters, a microorganism fermentatively synthesizing polyesters utilizing the gene, and a process for producing polyesters using the microorganism.

### BACKGROUND ART

At present, many kinds of microorganisms are known to accumulate polyesters such as polyhydroxyalkanoates (hereinafter referred to briefly as PHA) as the energy storage materials within cells. A representative example of the polyester is poly-3-hydroxybutyric acid (hereinafter referred to briefly as P(3HB)), which is a homopolymer of 3-hydroxybutyric acid (hereinafter referred to as 3HB for short). It was first discovered in Bacillus megaterium (M. Lemoigne, Ann. Inst. Pasteur, 39, 144 (1925)). P(3BH) is a thermoplastic polymer and is biodegradable in the natural environment and, thus, has recently attracted attention as an ecofriendly plastic. However, P(3HB) is high in crystallinity, and stiff and brittle material, so that the range of practical application thereof is limited. Therefore, research works have been undertaken to improve these properties.

Among others, a technology of producing a copolymer made of 3-hydroxybutyric acid (3HB) and 3-hydroxyvaleric acid (hereinafter referred to briefly as 3HV) (hereinafter such copolymer is referred to as P(3HB-co-3HV)) is disclosed (Japanese Kokai Publication Sho-57-150393 and Japanese Kokai Publication Sho-59-220192) . This P(3HB-co-3HV) is rich in flexibility as compared with P(3HB), hence was expected to have a broader application range. In actuality, however, P(3HB-co-3HV) shows only slight changes in physical properties even when the molar fraction of 3HV is increased. In particular, the flexibility can not be improved to the amount required for use in films and the like. Thus, it has been used only in the field of rigid shaped articles such as shampoo bottles and disposable razor grips.

In recent years, studies have been made concerning the copolymer consisting of two components 3HB and 3-hydroxyhexanoic acid (hereinafter referred to as 3HH for short) (hereinafter such copolyesters are referred to as P(3HB-co-3HH) for short) and the technology of producing it (Japanese Kokai Publication Hei-05-93049 and Japanese Kokai Publication Hei-07-265065). According to these patent documents, this technology of producing P(3HB-co-3HH) comprises fermentative production thereof from fatty acids, such as oleic acid, or oils or fats, such as olive oil, using Aeromonas caviae isolated from soil. Studies concerning the properties of P(3HB-co-3HH) have also been made (Y. Doi, S. Kitamura, H. Abe, Macromolecules, 28, 4822-4823 (1995)). According to this report, when A. caviae is cultured using fatty acids of not less than 12 carbon atoms as the only carbon source, P(3HB-co-3HH) with a 3HH fraction of 11 to 19 mole percent can be fermentatively produced. It has been revealed that the properties of such P(3HB-co-3HH) change from hard and brittle gradually to soft and flexible, to an extent exceeding the flexibility of P(3HB-co-3HV), with the increase in molar fraction of 3HH. However, the above method of production is low in productivity, namely the cell productivity is 4 g/L and the polymer content is 30%. Therefore, methods capable of attaining higher productivity for practical use have been searched for.

A PHA synthase gene has been cloned from Aeromonas caviae, which is a producible strain of P(3HB-co-3HH) (Japanese Kokai Publication Hei-10-108682; T. Fukui, Y. Doi, J. Bacteriol., vol. 179, No. 15, 4821-4830 (1997)). This gene was introduced into Ralstonia eutropha (formerly Alcaligenes eutrophus), and cultivation was carried out using the resulting transformant and a vegetable oil as the carbon source, whereby a content in cells of 4 g/L and a polymer content of 80% were attained (T. Fukui et al., Appl. Microbiol. Biotechnol., 49, 333 (1998)). A method of producing P(3HB-co-3HH) using bacteria, such as Escherichia coli, or a plant as the host has also been disclosed, without describing any productivity data, however (WO 00/43525, for example).

The above-mentioned polymer P(3HB-co-3HH) can be given a wide range of physical properties, from properties of rigid polymers to properties of flexible polymers, by changing the molar fraction of 3HH and therefore can be expected to be applicable in a wide range, from television boxes and the like, for which rigidity is required, to yarns, films and the like, for which flexibility is required. However, the production methods mentioned above are still poor in the productivity of P(3HB-co-3HH). There is no other way but to say that they are still unsatisfactory as practical production methods of P(3HB-co-3HH).

Recently, Kichise et al. reported a method, comprising modifying A. caviae-derived P(3HB-co-3HH) synthase by in vitro enzyme evolution system and then introducing it into Escherichia coli, to construct an Escherichia coli capable of accumulating more amount of P(3HB-co-3HH) than a wild-type gene-introduced Escherichia coli (T. Kichise et al., Appl. Environ. Microbiol. 68, 2411-2419 (2002)). In the report, E2-50 strain and T3-11 strain, in which specific activities of P(3HB-co-3HH) synthase were enhanced, were obtained. E2-50 is a mutant of P(3HB-co-3HH) synthase, in which serine is substituted for 149th amino acid asparagine, and T3-11 is one, in which glycine is substituted for 171st aspartic acid. Specific activity of the mutated enzyme in E2-50 strain is enhanced to about 1.5 times as high as that of wild type Escherichia coli, whereas that of T3-11 strain is enhanced to about 1.2 times. But in general, Escherichia coli cannot utilize fats and oils, which are low-cost carbon sources. Furthermore, it does not contain condensing enzymes of acetyl-CoA, which is necessary for a route to synthesize a component unit of P(3HB-co-3HH). Therefore, a low-cost and efficient production system cannot be attained unless other genes providing fats and oils-decomposing ability or substrate-supplying ability are otherwise introduced. Furthermore, Amara et al. obtained a mutant the specific activity of which increased fivefold, but polymer productivity was only slightly increased in the case where glucose was used as a carbon source (Amara AA. et al., Appl. Microbiol. Biotechnol. 59, 477-482 (2002)). In Escherichia coli, a mutated polymer synthase with increased activity cannot express its own abilities, and Escherichia coli cannot directly utilize vegetable oils and fats, which are low-cost carbon sources. Therefore, Escherichia coli lacks usefulness as a host for polymer production in economical viewpoint.

In a study of the production of biodegradable polyesters, Leaf et al. used yeast high in cell productivity as the host (Microbiology, vol. 142, pp. 1169-1180 (1996)). Thus, the polyester synthase gene of Ralstonia eutropha was introduced into Saccharomyces cerevisiae, a kind of yeast, the thus-produced transformant was cultured using glucose as the carbon source, and the accumulation of P(3HB) was confirmed. However, polymer content achieved in this study resulted in as low as 0.5% and the polymer was stiff and brittle P(3HB). Other studies also have been made on the production of copolymers containing a monomer unit having 5 or more carbon atoms, by expressing, in yeast Saccharomyces cerevisiae, a polyester synthase gene derived from Pseudomonas aeruginosa in the presence of fatty acids as a carbon source. But the polymer content achieved also resulted in as low as 0.5% in this case (Poirier Y. et al., Appl. Microbiol. Biotechnol. 67, 5254-5260 (2001)). Furthermore, other studies have been made on the production of polyester using oleic acid as a carbon source, by expressing and targeting, into peroxisomes of yeast Pichia Pastoris, a polyester synthase gene derived from the same Pseudomonas species. These studies showed that 1% weight of polymer was accumulated per dried cell unit (Poirier Y. et al., FEMS Microbiology Lett., vol. 207, pp.97-102 (2002)). But such low accumulation content is quite insufficient.

Yeast is known to grow fast and be high in cell productivity. Among them, yeasts belonging to the genus Candida attracted attention as single cell proteins in the past and, since then, studies have been made on the production of cells thereof for use as feeds using normal-paraffins as carbon sources. Further, in recent years, host-vector systems for the genus Candida have been developed, and the production of substances using the recombinant DNA technology has been reported (Kagaku to Seibutsu (Chemistry and Living organisms), vol. 38, No. 9, 614 (2000). When Candida utilis is used as the host, the α-amylase productivity is as high as about 12.3 g/L. Microorganisms of the genus Candida having such high substance productivity are expected to serve as hosts for polymer production. Furthermore, cells thereof can be separated from the culture fluid with ease as compared with bacteria and, thus, the polymer extraction and purification steps can be facilitated.

Thus, a method of producing P(3HB-co-3HH) having good physical properties using yeast belonging to the genus Candida, and the like, has been developed, but further improvement has been requested for polymer productivity (WO 01/88144). As one method for increasing polymer productivity per cell, there may be mentioned a method comprising increasing the amount of the polymer synthase in a cell. As examples of methods for increasing the amount of polymer synthase in a cell, there may be mentioned a method comprising using a strong promoter, a method comprising using a high-copy-number plasmid, a method comprising introducing a large number of enzyme expression units into a plasmid or chromosomes, or the like method. It has been known, however, that increase of the molecular number of enzymes per cell leads to decrease in the molecular weight of the product polymer (Sim S. J. et al., Nature Biotechnology, vol. 15, pp. 63-67 (1997); and Gerngross T. U., Martin D. P., Proc. Natl. Acad. Sci. U.S.A, vol. 92, pp. 6279-6283 (1995)). Because the molecular weight of the polymer remarkably affects properties of a biodegradable polymer, methods, which are able to increase productivity without decrease of molecular weight, are longed for.

### SUMMARY OF THE INVENTION

In view of the above-mentioned state of the art, it is an object of the present invention to provide a gene coding for a mutated PHA synthase which can be expressed in yeast in a functional and efficient manner, a yeast transformant transformed by a gene expression cassette comprising the gene, and a method for producing polyesters such as P(3HB-co-3HH) having biodegradability and good physical properties which comprises cultivating the transformant obtained in the above manner.

The present inventors made various investigations and, as a result, found that when genes, which code for amino acid sequences of a mutated Aeromonas caviae-derived PHA synthase wherein a specific mutation has been introduced into its amino acid sequence, are constructed, a gene expression cassette is constructed by joining a promoter and terminator capable of substantially functioning in yeast to each of those genes, the resulting gene expression cassette is further introduced into yeast and the resulting transformant is cultivated, a polyester can be produced and recovered from the culture in such a manner that very high productivity can be expected.

Thus, the present invention relates to use of a gene coding for a mutated Aeromonas caviae-derived PHA synthase in yeast.

Particularly, the present invention relates to a gene expression cassette

which comprises a gene coding for a mutated PHA synthase, and a promoter and a terminator both capable of functioning in yeast,

said gene coding for a mutated PHA synthase being obtainable by applying at. least one of the following amino acid substitutions from (a) to (h) to an Aeromonas caviae-derived PHA synthase having the amino acid sequence shown under SEQ ID NO:1;
(a) substitution of Ser for Asn-149
(b) substitution of Gly for Asp-171
(c) substitution of Ser or Gln for Phe-246
(d) substitution of Ala for Tyr-318
(e) substitution of Ser, Ala or Val for Ile-320
(f) substitution of Val for Leu-350
(g) substitution of Thr, Ser or His for Phe-353
(h) substitution of Ile for Phe-518.

Moreover, the invention relates to a gene,

which is obtainable by adding a DNA coding for a peroxisome-targeting signal to a gene coding for the mutated PHA synthase.

In a preferred embodiment thereof, the invention relates to the gene as mentioned above

wherein the peroxisome-targeting signal comprises the amino acid sequence shown under SEQ ID NO:2 or SEQ ID NO:3, and more preferably to the gene as mentioned above

wherein the peroxisome-targeting signal-encoding DNA has the nucleotide sequence shown under SEQ ID NO:4 or SEQ ID NO:5.

In another preferred embodiment, the invention relates to the gene as mentioned above

wherein at least one CTG codon in the gene coding for an Aeromonas caviae-derived PHA synthase has been modified into TTA, TTG, CTT, CTC or CTA.

The present invention also relates to a gene expression cassette which comprises the above gene, a promoter and a terminator both capable of functioning in yeast.

The present invention further relates to a transformant which is resulted from transformation of at least one gene expression cassette above-mentioned into yeast, and preferably relates to the above-mentioned transformant, wherein the yeast is Candida maltosa.

Furthermore, the invention relates to a method for producing polyesters, which comprises cultivating the above transformant and recovering the product polyester from the culture obtained, and preferably relates to the above-mentioned method, wherein the polyester is a copolyester obtainable by copolymerization of 3-hydroxybutyric acid represented by the following formula (1):

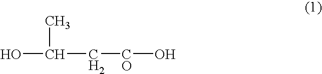

and 3-hydroxyhexanoic acid represented by the following formula (2):

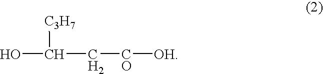

### DETAILED DESCRIPTION OF THE INVENTION

In the following, the present invention is described in detail.

(1) A Useful Mutant of Polyester Synthase

In the present invention, a mutated PHA synthase is used as a useful mutant, which is obtainable by applying at least one of the following amino acid substitutions from (a) to (h) to an Aeromonas caviae-derived PHA synthase having the amino acid sequence shown under SEQ ID NO:1;
(a) substitution of Ser for Asn-149
(b) substitution of Gly for Asp-171
(c) substitution of Ser or Gln for Phe-246
(d) substitution of Ala for Tyr-318
(e) substitution of Ser, Ala or Val for Ile-320
(f) substitution of Val for Leu-350
(g) substitution of Thr, Ser or His for Phe-353
(h) substitution of Ile for Phe-518.

In this description, for example, “Asn-149” means asparagine located at 149th position in the amino sequence, and amino acid substitution (a) means a conversion of asparagine located at 149th position into serine.

As a method to obtain the above mutant, there may be mentioned the following methods or the like. Various methods has been known to obtain modified mutant with improved properties such as enzyme activity, substrate specificity and/or thermal stability, by modifying amino acid sequences of polyester synthases derived from bacteria such as Aeromonas caviae. Especially, means utilizing molecular evolution technology (Japanese Kokai Publication 2002-199890) and the like are highly useful because desired mutants can be obtained in a short time. It is also possible to identify useful amino acid mutations on the basis of an enzyme conformation or an expectable conformation thereof by computing, for example, by using program Shrike (Japanese Kokai Publication 2001-184831) and the like.

It is not confirmed until actual applications whether thus-identified amino acid substitution (mutation) shows expected effects for the objective host or not. It is because the mutants of the above-mentioned T. Kichise et al. Appl. Environ. Microbiol. 68, 2411-2419 (2002) or Amara A A. et al., Appl. Microbiol. Biotechnol. 59, 477-482 (2002), which are obtained from Escherichia coli by means utilizing molecular evolution technology, are those most adaptable for the growing condition of Escherichia coli used for obtaining said mutants, and there is no guarantee that the mutants can adapt to different growing conditions, still less a different cell species. And it is also because mutants constructed by computing are not sufficient yet in accuracy.

Furthermore, combinations of mutations obtained by utilizing molecular evolution or the like technology makes it possible to give further suitable mutants. Therefore, mutants obtainable by applying at least two of the above amino acid substitutions from (a) to (h) are also favorably used.

(2) Construction of Mutated Genes for Yeast As a gene coding for the mutant obtainable by the method described in (1), there may be mentioned, for example, a gene obtainable by applying a DNA mutation corresponding to the amino acid substitution mentioned in (1) to the gene coding for an Aeromonas caviae-derived PHA synthase having the amino acid sequence shown under SEQ ID NO:1, and the like genes. Preferable one is a gene obtainable by introducing a DNA mutation corresponding to the above amino acid substitution mentioned in (1) into a PHA synthase gene, which is adapted to yeast.

Such a PHA synthase gene, which is adapted to yeast, is not particularly restricted, but for example, there may be mentioned ORF2 (shown under SEQ ID NO:3 in WO 01/88144), which is a gene coding for, in Candida maltosa, the same amino acid sequence as the gene coding for an Aeromonas caviae-derived PHA synthase, and the like genes.

Namely, use of the bacterial gene as it is may lead to occurrence, in some host yeasts, of some abnormality in translation of genetic codes in some instances. For example, CTG codon is translated into serine, not into leucine, in Candida maltosa (H. Sugiyama et al., Yeast, 11, 43-52 (1995)). Such problem can be solved by using a modified gene wherein at least one CTG codon contained in the latter gene has been converted to another codon corresponding to leucine (TTA, TTG, CTT, CTC, or CTA) in advance. Furthermore, considering the frequency of codon usage composing a gene of the host in use, a gene, which has a modification converting a codon to another codon with high usage, may be constructed and utilized.

In introduction of the amino acid substitution (mutation) described in (1) into the above-mentioned PHA synthase gene, which is adapted to yeast, a mutation of a codon may be introduced into an objective site by using site-directed mutagenesis. The method to introduce a mutation into an amino acid sequence of a gene in a site-specific manner may be carried out by using the recombinant DNA method, the PCR method or the like. For example, when appropriate restriction sequences are present at the both sides of the objective site in the PHA synthase gene into which a mutation is introduced, it may be carried out by a cassette mutation method, which comprises cleaving the sequences by the corresponding restriction enzymes, removing the region containing the site into which an introduction of a mutation is required, and then inserting a DNA fragment which has a mutation introduced into only the objective site by chemical synthesis or the like. Introduction of a site-specific mutation by PCR may be carried out by amplifying one side of the PHA synthase gene with a primer for mutation, which has the objective mutation introduced into the site into which an introduction of the mutation is required, and a primer for amplification, which does not have a mutation comprising one terminal sequence of said gene, amplifying another side with a primer for mutation, which has a complementary sequence to the above primer for mutation, and a primer for amplification, which does not have a mutation comprising another terminal sequence of said gene, annealing the obtained two amplified fragments, and further carrying out PCR using the above two kinds of primers for amplification. The constructs obtained by a site-specific manner are confirmed by determination of the nucleotide sequences thereof. The determination of the nucleotide sequences may be carried out by a known manner in the art using an automatic nucleotide sequence analyzer or the like.

Generally, in fermentative production of a polyester using yeast, glucose, fats and oils, fatty acids and the like, which can be utilized by the yeast, can be used as carbon sources, without any particular restriction. Particularly, in fermentative polyester production using fats and oils, fatty acids, n-paraffins or the like as carbon sources, these carbon sources are metabolized via the β oxidation cycle, and the metabolic intermediates in the β oxidation cycle are utilized as substrates for polyester synthesis with good efficiency (T. Fukui, Y. Doi, J. Bacteriol., 179, No. 15, 4821-4830 (1997); Q. Ren et al., J. Bacteriol., 182, No. 10, 2978-2981 (2000)). Since the β oxidation in yeast is carried out in peroxisomes, which are intracellular microbodies, localization, in peroxisomes, of an enzyme involved in polyester synthesis is favorable for efficient polyester synthesis.

The proteins to be transferred to peroxisomes are synthesized on free ribosomes and, owing to the function of a peroxisome-targeting signal presenting in the protein sequences, they are transferred to peroxisomes (S. Subramani, J. Membrane Biol., 125, 99-106 (1992); Y. Itai, Kagaku to Seibutsu 35, No. 10, 687-695 (1997); E. H. Hettema, Biochim. Biophys. Acta, 1451, 17-34 (1999)).

Therefore, in the present invention, addition of a DNA coding for such peroxisome-targeting signal to a gene coding for an enzyme involved in polyester synthesis, namely the gene coding for a mutated PHA synthase, is preferable, and, by this addition, it becomes possible to localize the enzyme involved in polyester synthesis, namely a mutated PHA synthase, in peroxisomes for efficient polyester synthesis.

Known as peroxisome-targeting signals occurring at the carboxyl terminus are sequences comprising three amino acid residues, namely “(serine/alanine/cysteine)-(lysine/arginine/histidine)-leucine”. The expression (serine/alanine/cysteine), for instance, as used herein means any one of serine, alanine and cysteine. For the targeting of the mutated PHA synthase toward peroxisomes, the addition of the above three-amino-acid sequence to the carboxyl terminus of the enzyme is sufficient. Among the peroxisome-targeting carboxyl-terminal signals, the most commonly known one, namely “serine-lysine-leucine” (hereinafter referred to as SKL for short) (SEQ ID NO:2) or “alanine-lysine-isoleucine” (hereinafter referred to as AKI for short) (SEQ ID NO:3), which is known as the peroxisome-targeting carboxyl-terminal signal in Candidas trpicalis (J. D. Aitchison et al., J. Biol. Chem., 266, 23197-23203 (1991)), is preferably added to the carboxyl terminus.

The nucleotide sequence corresponding to such amino acid sequence is not particularly restricted. In the case of SKL, the nucleotide sequence shown under SEQ ID NO:4 can be utilized and, in the case of AKI, that shown under SEQ ID NO:5 can be used, for example.

Further, sequences occurring in the vicinity of the N terminus and comprising 9 amino acid residues, namely “(arginine/lysine)-(leucine/valine/isoleucine)-(5 amino acid residues)-(histidine/glutamine)-(leucine/alanine)”, are also known as peroxisome-targeting signals. By inserting and adding these sequences into the amino acid sequence. of the mutated PHA synthase, it is possible to cause localization of the enzyme in peroxisomes.

In order to add a DNA coding for such a peroxisome-targeting signal as mentioned above to the gene coding for the mutated PHA synthase, chemical synthesis, the PCR method or the like method can be used.

(3) Gene Expression Cassette Construction

The gene expression cassette of the present invention comprises the mutated gene of (2), a promoter and a terminator both capable of functioning in yeast.

For the above mutated gene of (2) to be expressed in yeast, it is necessary that such DNA sequences as a promoter, the upstream activating sequence (UAS), etc. occur on the 5′ upstream of the gene and such DNA sequences as poly(A) additional signal, terminator, etc. on the 3′ downstream of the gene. When an appropriate site satisfying the above conditions occurs on a yeast chromosome, the gene in question may be directly inserted therein. Alternatively, the gene may be inserted into a plasmid having an appropriate promoter and terminator so that yeast may be transformed by the resulting plasmid.

In the practice of the present invention, a gene expression cassette is preferably constructed by ligating a promoter to the gene on the 5′ upstream thereof and a terminator on the 3′ downstream thereof so that the cassette may be used in transforming yeast.

Any promoter and terminator sequences may be used provided that they can function in yeast, which is to be a host. While, among the promoters, there are ones causing constitutive expression and ones causing inducible expression, either type of promoter may be used. In the practice of the present invention, it is desirable that, when Candida maltosa is used as a host, the promoter and terminator can function in Candida maltosa, hence the promoter and terminator be derived from Candida maltosa. More preferably, the Candida maltosa ALK1, ALK2 or ALK5 gene-derived promoter and the ALK1 gene-derived terminator are utilized.

Thus, for example, the promoter ALKlp (WO 01/88144) of the Candida maltosa ALK1 gene (GenBank D00481), the promoter ALK5p (SEQ ID NO:6) of the ALK5 gene, and the like, can be used as the promoter. Furthermore, a promoter (Kogure, et al., Abstract of the lectures of Japan Society for Bioscience, Biotechnology, and Agrochemistry, p. 191 (2002)), which is improved in its promoter activity by an addition of plural of ARR (alkane responsible region) sequences on the upstream of these promoters, can be utilized (SEQ ID NO:7). The terminator ALK1t (WO 01/88144) of the Candida maltosa ALK1 gene and the like terminator can be used as the terminator.

The nucleotide sequences of the above promoters and/or terminators each may be the nucleotide sequences in which one or a plurality of nucleotides may have undergone deletion, substitution and/or addition provided that they can function in Candida maltosa. In this description, the expression “the nucleotide sequences in which one or a plurality of nucleotides may have undergone deletion, substitution and/or addition” means the nucleotide sequences with deletion, substitution, and/or addition, wherein such number of nucleotides to be capable of being deleted, substituted and/or added by the known method in the prior art, such as “Protein-Nucleic acid-Enzyme, a special issue, gene-amplifying PCR method TAKKAJ 35(17), 2951-3178(1990)” or “PCR technology(1990), edited by Henry A. Erlich, translated by Ikunoshin Kato”, etc. are deleted., substituted and/or added.

The promoter is ligated to the 5′ upstream of the gene coding for the mutated Aeromonas caviae-derived PHA synthase with an added DNA coding for a peroxisome-targeting signal, and the terminator is ligated to the 3′ downstream of the gene coding for the mutated PHA synthase with the added DNA coding for a peroxisome-targeting signal.

The vector to be used in construction of the gene expression cassette may be any of those capable of autonomous replication in Escherichia coli. It may further have a region capable of autonomous replication in yeast. The vector capable of autonomous replication in yeast is maintained in microbial cells. It is also possible to integrate the gene expression cassette into a chromosome. As an example of such vector, pUTUl capable of autonomous replication in Candida maltosa can be used (M. Ohkuma, et al., J. Biol. Chem., vol. 273, 3948-3953 (1998)).

The method of constructing the gene expression cassette according to the present invention by joining the promoter and terminator to the structural gene is not particularly restricted. Except for ones represented in the example section to be described below, of the present invention, the PCR method can be utilized in order to form appropriate restriction sites. The method described in WO 01/88144 can be used, for example.

(4) Host

The “yeast” so referred to herein is not particularly restricted. Thus, usable are yeasts deposited with organism depositories (e.g. IFO, ATCC, etc.) and belonging to such genera as Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkera, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma.

The yeast to be used as the transformant host in the practice of the present invention is not particularly restricted but, among them, preferably is one belonging to the genus Candida or Yarrowia, more preferably Candida maltosa or Yarrowia lipolytica, particularly preferably Candida maltosa.

Among the yeasts usable as the transformant hosts, the Candida maltosa AC16 strain has been internationally deposited with the National Institute of Advanced Industrial Science and Technology, Central 6, 1-1-1 Higashi, Tsukuba, Ibaraki, Japan, on Nov. 15, 2000 under the accession number FERM BP-7366.

(5) Transformant Preparation

The transformant of the present invention is resulted from introduction of at least one gene expression cassette of (3) into yeast.

The gene expression cassette recombinant vector involved in polymer synthesis can be introduced into yeast in the conventional manner, using, for example, the calcium phosphate method (Lederberg, E. M. et al., J. Bacteriol., 119, 1072 (1974)), the electroporation method (Current Protocols in Molecular Biology, vol. 1, 1.8, p. 4 (1994)), or the like. Commercially available transformation kits such as Fast Track TM-Yeast Transformation Kit SM (Geno Technology) can also be utilized.

As an example, the Candida maltosa CHA1 strain (S. Kawai, et al., Agric. Biol. Chem., vol. 55, 59-65 (1991)) can be used as the host. By transforming this strain using a plasmid vector containing the gene expression cassette involved in polymer synthesis, and the like, by the transformation method mentioned above, it is possible to produce Candida maltosa transformants having a plasmid such as pARR-ORF2S, represented in the example section to be described below, or the like plasmid.

Each of the transformant AC16 pUTA-149NS×2 (originally deposited domestically on May 8, 2003 under the accession number FERM BP-10019, and then transferred to the Budapest Treaty depositry), which is transformed by the plasmid pARR-149NS×2, and the plasmid pARR-149NS/171DG×2 (deposited internationally on Apr. 27, 2004 under the accession number FERM BP-10017 to the Budapest Treaty depositry) has been internationally deposited with the National Institute of Advanced Industrial Science and Technology, Central 6, 1-1-1 Higashi, Tsukuba, Ibaraki, Japan.

(6) Polyester Production

In accordance with the polyester production method of the present invention, a polyester is recovered from the culture obtainable by cultivating the above-mentioned transformant of the present invention.

Namely, the polyester production of the present invention can be carried out by adding the transformant of the present invention into a medium, culturing the same, and then recovering a polyester from the cultured cells or the culture obtained. The cultivation temperature is within a temperature range in which the organism can grow, preferably 15° C. to 40° C., more preferably 20° C. to 40° C., and still more preferably 28° C. to 34° C. The cultivation time is not particularly restricted but, for example, 1 to 7 days are preferred in the case of a batch culture. Moreover, a continuous culture can be also utilized.

The medium is not particularly restricted provided that yeast can utilize. Media containing a carbon source, a nitrogen source, an inorganic salt, other organic nutrient sources and the like can be used, for example.

The carbon source is not particularly restricted provided that yeast can assimilate, carbohydrates, fats and oils, fatty acids, n-paraffins and the like can be used. As the carbohydrates, there may be mentioned, for example, glucose, sucrose and glycerol, etc. As the fats and oils, there may be mentioned, for example, rapeseed oil, coconut oil, palm oil and palm kernel oil, etc. As the fatty acids, there may be mentioned, for example, hexanoic acid, octanoic acid, decanoic acid, lauric acid, oleic acid, palmitic acid, linolic acid, linolenic acid, myristic acid, and like saturated and unsaturated acids, as well as esters and salts of these fatty acids and other fatty acid derivatives. As the n-paraffins, there may be mentioned, for example, dodecane and tetradecane, etc.

When the promoter expression is of the inducible type, an inducer (e.g. alcohols and the like) is to be added appropriately. In some instances, the inducer may serve as the main carbon source.

As an example, Candida maltosa can be cultivated using fats or oils as carbon sources. In the case of yeast, which cannot assimilate fats or oils efficiently or at all, improvements can be achieved by adding lipase to the medium. Furthermore, yeast can be provided with the ability to assimilate fats and oils by transformation with a lipase gene.

As the nitrogen source, there may be mentioned, for example, ammonia, ammonium chloride, di-ammonium sulfate, ammonium phosphate, and other ammonium salts, as well as peptone, meat extract, yeast extract, and the like.

As the inorganic salts, there may be mentioned, for example, potassium dihydrogenphosphate, di-potassium hydrogenphosphate, magnesium phosphate, magnesium sulfate, sodium chloride, and the like.

The other organic nutrient sources include, for example, amino acids such as glycine, alanine, serine, threonine, proline and the like; vitamins such as vitamin B1, vitamin B12, biotin, nicotinamide, pantothenic acid, vitamin C and the like; and the like.

In the practice of the present invention, the polyester is recovered from yeast cells using the following method, for instance. After completion of cultivation, cells are separated from the culture fluid using a centrifuge and the like and the cells are washed with distilled water and methanol or the like, and then dried. The polyester is extracted from these dried cells using an organic solvent such as chloroform and the like. The cell fraction is removed from the organic solvent solution containing the polyester by filtration and the like. A poor solvent, such as methanol, hexane or the like, is added to the filtrate to cause the polyester to precipitate out. The precipitate polyester is separated from the supernatant by filtration or centrifugation, and dried. The polyester can be thus recovered. Since yeast cells are used as polyester producer cells in accordance with the present invention, such simple and easy methods of separation and recovery as mentioned above can be utilized.

The polyester obtained is analyzed by, for example, gas chromatography, nuclear magnetic resonance spectrometry and/or the like. Molecular weights can be determined by GPC method. Recovered dried polymers are dissolved in chloroform, and then this solution may be analyzed by Shimadzu GPC system equipped with Shodex K805L (product of Showa Denko) using chloroform as a mobile phase. Commercial standard polystyrene and the like may be used as the standard molecular weight sample.

As described above, in the present invention, by constructing a transformant of Candida maltosa using a gene expression cassette which comprises a gene coding for a mutated Aeromonas caviae-derived PHA synthase, which is improved in its enzymatic activity, or a gene resulting from addition of a DNA coding for a peroxisome-targeting signal to a gene coding for a mutated Aeromonas caviae-derived PHA synthase, which is improved in its enzymatic activity, together with a terminator and a promoter both capable of functioning in yeast, and then cultivating the same transformant, polyesters can be synthesized efficiently.

In the present invention, as polyesters, the copolyester P(3HB-co-3HH), which results from copolymerization of 3-hydroxybutyric acid represented by the general formula (1) and 3-hydroxyhexanoic acid represented by the general formula (2), can be preferably produced.

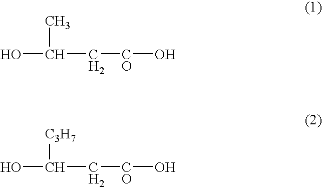

### BRIEF DESCRIPTION OF THE DRAWINGS

FIG. 1 is a schematic diagram of construction of pSTARR from pUAL1, and a simple diagram of each plasmid, as described in the example section.

FIG. 2 is a schematic diagram of construction of pARR-149NS×2 and the like from pSTARR, and a simple diagram of each plasmid, as described in the example section.

### BEST MODE FOR CARRYING OUT THE INVENTION

The following examples illustrate the present invention more specifically. These examples are, however, by no means limitative of the technical scope of the present invention.

### EXAMPLE 1

Synthesis and Modification of PHA Synthase Gene

Based on the amino acid sequences of an Aeromonas caviae-derived PHA synthase (T. Fukui, et al., FEMS Microbiology Letters, vol. 170, 69-75 (1999)) shown under SEQ ID NO:1, the corresponding PHA synthase gene was synthesized.

Since Candida maltosa is yeast translating the CTG codon into serine, not leucine, CTG was not assigned to the leucine codon. That codon with high codon usage in Candida maltosa was preferentially selected as the codon corresponding to each amino acid. For codon usage, Klaus Wolf: Nonconventional Yeast in Biotechnology (published by Springer) was referred to. In this way, the PHA synthase gene (ORF2) (SEQ ID NO:3 in WO 01/88144) was designed and chemically synthesized. Then, the product was cloned into. pUCNT (described in WO 94/03613).

Next, pUCNT, in which a PHA synthase gene was cloned, was amplified using SEQ ID NOs:8 and 9, as primers for PCR, for substitution of serine for asparagine, which is 149th amino acid of said PHA synthase. Pfu polymerase (product of Stratagene) was used for the.amplification. After 18 cycles of PCR (each cycle comprising: 1 minute at 96° C., 1 minute at 60° C., 11 minutes at 68° C.), a restriction enzyme DpnI was added to thereby cleave a template plasmid, then transforming E. coli JM109 strain, and recovering a plasmid from the resultant transformant. Using primers of SEQ ID NOs:10 to 14, the nucleotide sequence thereof was analyzed. The nucleotide sequence was determined by using DNA sequencer 310 Genetic Analyzer (product of PERKIN ELMER APPLIED BIOSYSTEMS). Thus, the gene coding for a mutated PHA synthase, named ORF2-149NS, in which gene mutation was introduced into an objective site, was constructed.

### EXAMPLE 2

Construction of an Expression Cassette Comprising a Gene for a Mutated PHA Synthase

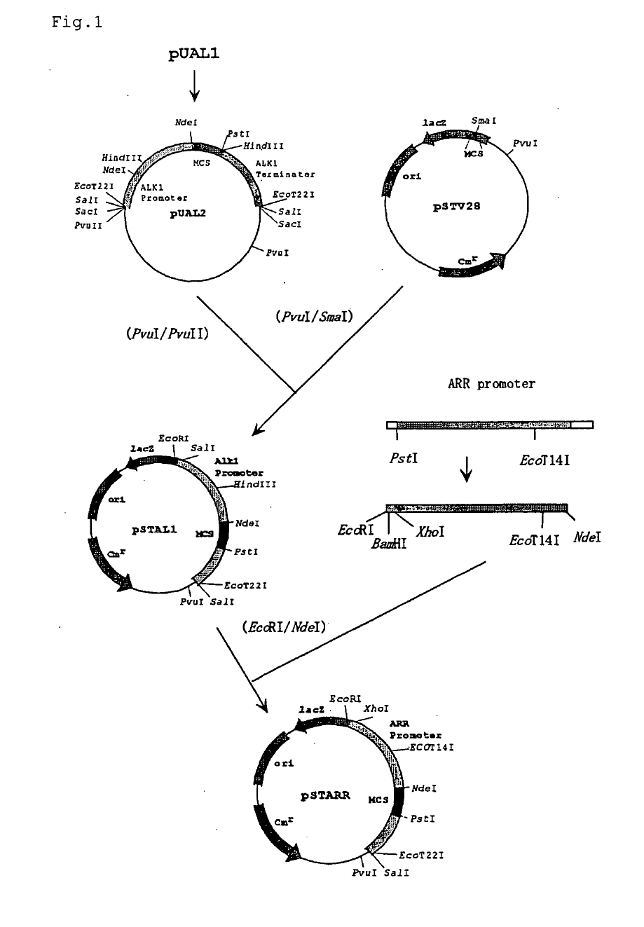

For causing expression of PHA synthase in Candida maltosa, it was decided that a Candida maltosa-derived promoter be ligated to the 5′ upstream of each gene, and a Candida maltosa-derived terminator to the 3′ downstream of each gene. It was decided to use the promoter ARRp, in which the ARR sequence was added to the upstream of Alk2 gene (GenBank X55881) promoter, as the promoter, and to ligate the Candida maltosa AlK1 gene (GenBank D00481) terminator ALK1t to the 3′ downstream of each. ARRp gene imparted from Tokyo University (SEQ ID No:15) was converted, so as to be digested with XhoI and NdeI, by ligating EcoRI-XhoI linker to the PstI site and the synthetic DNA shown under SEQ ID No:16 to the EcoT14I site. The vector pUAL1 (described in WO 01/88144) was cleaved by EcoRI, then converted to be blunt-ended form, and subjected the resultant to ligation, to construct pUAL2, in which EcoRI-cleaved site was removed. The pUAL2 was digested with PvuI/PvuII, and ligated to the PvuI SmaI site of pSTV28 (Takara Shuzo) to construct pSTALl. This pSTAL1 was digested with EcoRI/NdeI, and ligated to the above-mentioned ARRp to construct pSTARR. FIG. 1 shows a schematic diagram of construction of pSTARR from pUAL1 and a schematic diagram of each plasmid.

It was decided that a peroxisome-targeting signal be added to the carboxy terminus of each of ORF2 and ORF2-149NS mentioned in Example 1 so that each of them may be expressed in Candida maltosa and targeted to peroxisomes. The amino acid sequence of Ser-Lys-Leu (SKL) was used as the peroxisome-targeting signal to be added to the carboxy terminus. With the ORF2 and ORF2-149NS as templates, which are cloned in pUCNT, ORF2S and ORF2S-149NS were constructed using SEQ ID NOs:17 and 18 as primers. These genes coding for a mutated PHA synthase were ligated at NdeI and PstI sites of pSTARR, to thereby construct pSTARR-ORF2S and pSTARR-ORF2S149NS.

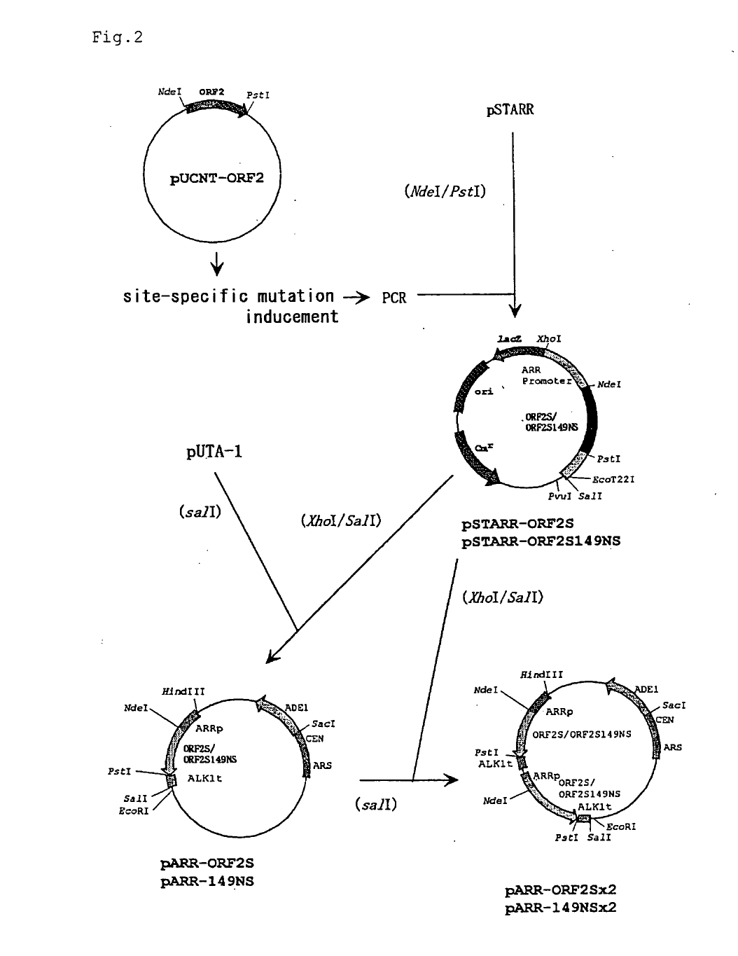

As a vector to finally ligate to the gene coding for a mutated PHA synthase, pUTA-1 (described in WO 01/88144) was used. An expression cassette was excised from pSTARR-ORF2S and pSTARR-ORF2S149NS with XhoI/SalI and ligated to SalI site of pUTA-1 to thereby construct pARR-ORF2S and pARR-149NS. Furthermore, the constructed pARR-ORF2S and pARR-149NS were digested with SalI, and expression cassettes, which were excised with XhoI/SalI from pSTARR-ORF2S and pSTARR-ORF2S-149NS, were ligated, to construct pARR-ORF2S×2 and pARR-149NS×2. FIG. 2 shows a schematic diagram of construction of pARR-149NS×2 and the like from pSTARR and a simple diagram of each plasmid.

### EXAMPLE 3

Transformant Construction

Unless otherwise specified, the reagents used in yeast cultivation were commercial products available from Wako Pure Chemical Industries. The host used was the Candida maltosa AC16 strain, which is a strain with the ADE1 gene disrupted and has been internationally deposited with the National Institute of Advanced Industrial Science and Technology International Patent Organism Depositary (accession number FERM BP-7366), and the plasmids comprising the above-mentioned gene expression cassettes of the present invention, namely pARR-ORF2S, pARR-149NS, pARR-ORF2S×2, and pARR-149NS×2, were respectively introduced into the host. Each plasmid constructed was introduced into the host by the electroporation technique. The gene transfer apparatus used was ELECTRO CELL MANIPULATOR 600 (product of BTX). The cuvettes used were BM 6200 cuvettes produced by BIO MEDICAL CORPORATION CO. LTD. Each plasmid (1 μl) was added to 100 μl of competent cells. 100 μl of the thus-prepared competent cell/plasmid solution was taken and poured into a cuvette, which was set on the pulse generator. Then, electric pulses were applied under the following conditions: electrostatic capacity 40 μF, resistance value 246 ohm, and voltage 1.9 kV. After pulse application, 1 ml of 1 M sorbitol was added to each cuvette and, after gentle mixing, the cuvette was allowed to stand at room temperature for 1 hour. After plasmid introduction, the cells were cultured on a selection plate (0.67 w/v % Yeast Nitrogen base without amino acid (product of Difco), 2 w/v % glucose, 2 w/v % agar), to give transformants. Among them, the one harboring the plasmid pARR-149NS×2 has been internationally deposited as AC16 pUTA-149NS×2 with the National Institute of Advanced Industrial Science and Technology International Patent Organism Depositary (FERM BP-10019).

### EXAMPLE 4

Polymer Production Using the Transformants

The Candida maltosa transformants resulting from introduction of the gene necessary for polymer production were cultivated in the following manner. YNB medium (0.67 w/v % Yeast Nitrogen base without amino acid, 2 w/v % Glucose) was used as a medium for preculture, and M2 medium (12.75 g/L ammonium sulfate, 1.56 g/L potassium dihydrogenphosphate, 0.33 g/L dipotassium hydrogenphosphate trihydrate, 0.08 g/L potassium chloride, 0.5 g/L sodium chloride, 0.41 g/L magnesium sulfate heptahydrate, 0.4 g/L calcium nitrate heptahydrate, and 0.01 g/L Iron(III) trichloride tetrahydrate) supplemented with 2 w/v % palm oil and 0.45 ml/L of trace elements dissolved in hydrochloric acid (1 g/mL Iron(II) sulfate heptahydrate, 8 g/mL zinc(II) sulfate heptahydrate, 6.4 g/mL manganese(II) sulfate tetrahydrate, and 0.8 g/mL cuprous(II) sulfate pentahydrate) was used as a production medium.

A 500-ml Sakaguchi flask containing 50 ml of the above medium for preculture was inoculated with 500 μl of a glycerol stock of each transformant and, after 20 hours of cultivation, the culture was inoculated into a 2-L Sakaguchi flask containing 300 mL of the production medium at an inoculum size of 10 v/v %. This was cultivated at an incubation temperature of 30° C. and a shaking speed of 90 rpm for 2 days. Cells were recovered from the culture fluid by centrifugation, suspended in 80 ml of distilled water, and disrupted using an ultrahigh pressure homogenizer (APV's Rannie 2000, at 15,000 Psi for 15 minutes), followed by centrifugation. The precipitate obtained was washed with methanol and then lyophilized.

The lyophilized cells were ground, 100 ml of chloroform was added thereto, and the mixture was stirred overnight for effecting extraction. The cells were removed by filtration, the filtrate was concentrated to 1 to 2 ml using an evaporator, and about 10 ml of hexane was added to the concentrate to cause the polymer to precipitate out. The cultivation results thus obtained are shown in Table 1. The molecular weights were measured as follows. The recovered dried polymer (10 mg) was dissolved in 5 ml of chloroform, and then solid matters were removed therefrom by filtration. This obtained solution was analyzed by Shimadzu GPC system equipped with Shodex K805L (300×8 mm, two columns were connected)(product of Showa Denko) using chloroform as a mobile phase. As the standard molecular weight sample, commercial standard polystyrene was used and the molecular weights were determined as weight-average molecular weight. The 3HH molar fraction was measured with NMR (JOEL, JNM-EX400).

TABLE 1

| | | 3HH | |
|---|---|---|---|
| | Polymer content | molar fraction | Molecular weight |
| Plasmid | (wt %) | (mol %) | (M.W.) |
| pARR-ORF2S | 2.1 | 15.4 | 450000 |
| pARR-ORF2Sx2 | 2.97 | 21.7 | 260000 |
| pARR-149NS | 7.1 | 23.8 | 710000 |
| pARR-149NSx2 | 12.8 | 28.4 | 455000 |

As shown in the above results, the yeast transformed by plasmids pARR-149NS and pARR-149NS×2 containing the expression cassette of the present invention, which comprises a mutated gene coding for a mutated PHA synthase, increased polymer content and 3HH molar fraction as compared with the case of the yeast transformed by plasmids pARR-ORF2S and pARR-ORF2S×2 containing an expression cassette comprising a gene coding for a wild-type PHA synthase. From these results, not only improving effect in activities of the mutated PHA synthases of the present invention was demonstrated, but also tendency of increase of the obtained PHA polymers was found. Thus, usefulness of the mutated PHA synthase of the present invention was demonstrated.

### EXAMPLE 5

Double-Mutant

A mutant, in which glycine was substituted for aspartic acid, 171st amino acid of the PHA synthase, was constructed according to the method described in Example 1. SEQ ID NOs:19 and 20 were used for the primers to introduce the mutation. In addition, a double-mutant, in which serine was substituted for 149th amino acid asparagine and glycine was substituted for 171st amino acid aspartic acid, was constructed by the same method as in Example 1, using ORF2-149NS, a gene coding for a mutated PHA synthase constructed in Example 1, as a template. Thus, ORF2-171DG, a gene coding for a mutated PHA synthase, and ORF2-149NS/171DG, a gene coding for a double-mutated PHA synthase, were completed.

Next, using these mutated genes, pSTARR-ORF2S171DG and pSTARR-ORF2S149NS/171DG were constructed according to the method described in Example 2, by adding a signal sequence using SEQ ID NOs:17 and 18 as primers, and cloning them into plasmid pSTARR. By introducing these mutated gene expression cassettes into pUTA-1 according to the method described in Example 2, plasmids pARR-171DG and pARR-149NS/171DG were constructed. Then, an expression cassette using the terminator LACt (SEQ ID NO:21) of LAC4 gene (GenBank M84410) in lieu of the terminator ALK1t was constructed. ALK1t in pSTARR-ORF2S171DG and pSTARR-ORF2S149NS/171DG were removed by PstI/SalI, and a plasmid, into which LAC4t amplified with primers shown under SEQ ID NOs:22 and 23 was introduced, was constructed instead. This expression cassette comprising a gene coding for a mutated PHA synthase, in which the terminator was substituted, was excised by XhoI/SalI, respectively, and then ligated to SalI site of plasmids pARR-171DG and pARR-149NS/171DG, to complete plasmids pARR-171DG×2 and pARR-149NS/171DG×2, into which two expression cassettes comprising a gene coding for a mutated PHA synthase were introduced. Among them, the plasmid pARR-149NS/171DG×2 has been internationally deposited with the National Institute of Advanced Industrial Science and Technology International Patent Organism Depositary (FERM BP-10017).

These plasmids were introduced into Candida Maltosa AC16 strain (FERM BP-7366) according to the method described in Example 3, and polymer production and analysis were carried out according to the method described in Example 4. The results are shown in Table 2, and were compared with that of Example 4.

TABLE 2

| | | 3HH | |
|---|---|---|---|
| | Polymer content | molar fraction | Molecular weight |
| Plasmid | (wt %) | (mol %) | (M.W.) |
| pARR-ORP2Sx2 | 2.97 | 21.7 | 1260000 |
| pARR-149NSx2 | 12.8 | 28.4 | 1455000 |
| pARR-171DGx2 | 4.5 | 23.0 | 330000 |
| pARR-149NS/ | 14.0 | 34.1 | 320000 |
| 171DGx2 | | | |

As shown in the above results, the yeast transformed by plasmids pARR-171DG×2 containing the expression cassette of the present invention, which comprises a mutated gene coding for a mutated PHA synthase, increased polymer content as compared with the case of using the yeast transformed by plasmid pARR-ORF2S×2 containing an expression cassette comprising a gene coding for a wild-type PHA synthase. Furthermore, it was revealed that the yeast transformed with the plasmid pARR-149NS/171DG×2 containing an expression cassette comprising a double-mutated gene coding for a double-mutated PHA synthase further increased polymer content as well as 3HH molar fraction. Thus, usefulness of the mutated PHA synthase of the present invention was demonstrated.

### INDUSTRIAL APPLICABILITY

The present invention made it possible to produce copolyesters resulting from copolymerization of a 3-hydroxyalkanoic acid(s) such as 3-hydroxybutyric acid and 3-hydroxyhexanoic acid each represented by the general formula (1) and (2) and having biodegradability and good physical properties in yeast efficiently.

1. A gene expression cassette
which comprises a gene coding for a mutated polyhydroxyalkanoic acid synthase, and a promoter and a terminator both capable of functioning in yeast,
said gene coding for a mutated polyhydroxyalkanoic acid synthase being obtainable by applying at least one of the following amino acid substitutions from (a) to (h) to an Aeromonas caviae-derived polyhydroxyalkanoic acid synthase having the amino acid sequence shown under SEQ ID NO:1;
(a) substitution of Ser for Asn-149
(b) substitution of Gly for Asp-171
(c) substitution of Ser or Gln for Phe-246
(d) substitution of Ala for Tyr-318
(e) substitution of Ser, Ala or Val for Ile-320
(f) substitution of Val for Leu-350
(g) substitution of Thr, Ser or His for Phe-353
(h) substitution of Ile for Phe-518.

2. A gene,
which is obtainable by adding a DNA coding for a peroxisome-targeting signal to a gene coding for a mutated polyhydroxyalkanoic acid synthase,
said gene being obtainable by applying at least one of the following amino acid substitutions from (a) to (h) to an Aeromonas caviae-derived polyhydroxyalkanoic acid synthase having the amino acid sequence shown under SEQ ID NO:1;
(a) substitution of Ser for Asn-149
(b) substitution of Gly for Asp-171
(c) substitution of Ser or Gln for Phe-246
(d) substitution of Ala for Tyr-318
(e) substitution of Ser, Ala or Val for Ile-320
(f) substitution of Val for Leu-350
(g) substitution of Thr, Ser or His for Phe-353
(h) substitution of Ile for Phe-518.

3. The gene according to claim 2,
wherein the peroxisome-targeting signal comprises the amino acid sequence shown under SEQ ID NO:2 or SEQ ID NO:3.

4. The gene according to claim 3,
wherein the peroxisome-targeting signal-encoding DNA has the nucleotide sequence shown under SEQ ID NO:4 or SEQ ID NO:5.

5. The gene according to claim 2,
wherein at least one CTG codon in the gene coding for an Aeromonas caviae-derived polyhydroxyalkanoic acid synthase has been modified into TTA, TTG, CTT, CTC or CTA.

6. A gene expression cassette
which comprises the gene according claim 2, a promoter and a terminator both capable of functioning in yeast.

7. A transformant
which is resulted from introduction of at least one gene expression cassette according to claim 1 into yeast.

8. The transformant according to claim 7,
wherein the yeast is Candida maltosa.

9. A method of producing polyesters,
which comprises cultivating the transformant according to claim 7 and recovering the product polyester from the culture obtained.

10. The method according to claim 9,
wherein the polyester is a copolyester obtainable by copolymerization of 3-hydroxybutyric acid represented by the following formula (1): 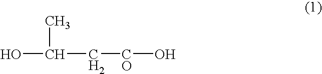
and 3-hydroxyhexanoic acid represented by the following formula (2):

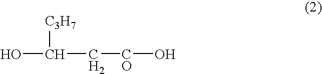

11. The gene according to claim 3,
wherein at least one CTG codon in the gene coding for an Aeromonas caviae-derived polyhydroxyalkanoic acid synthase has been modified into TTA, TTG, CTT, CTC or CTA.

12. The gene according to claim 4,
wherein at least one CTG codon in the gene coding for an Aeromonas caviae-derived polyhydroxyalkanoic acid synthase has been modified into TTA, TTG, CTT, CTC or CTA.

13. The gene according to claim 5,
wherein at least one CTG codon in the gene coding for an Aeromonas caviae-derived polyhydroxyalkanoic acid synthase has been modified into TTA, TTG, CTT, CTC or CTA.

14. A gene expression cassette
which comprises the gene according to claim 3, a promoter and a terminator both capable of functioning in yeast.

15. A gene expression cassette
which comprises the gene according to claim 4, a promoter and a terminator both capable of functioning in yeast.

16. A gene expression cassette
which comprises the gene according to claim 5, a promoter and a terminator both capable of functioning in yeast.

17. A transformant
which is resulted from introduction of at least one gene expression cassette according to claim 6 into yeast.

18. The transformant according to claim 17,
wherein the yeast is Candida maltosa.

19. A method of producing polyesters,
which comprises cultivating the transformant according to claim 17 and recovering the product polyester from the culture obtained.

20. A method of producing polyesters,
which comprises cultivating the transformant according to claim 18 and recovering the product polyester from the culture obtained.
